(19) United States
(12) Patent Application Publication
(10) Pub. No.: US 2006/0173062 A1
(43) Pub. Date: Aug. 3, 2006

(54) Use of selective cyclooxygenase-2 inhibitors for the treatment of endometriosis

(76) Inventors: Judith A. Boice, Yardley, PA (US); Susan P. Rohrer, Scotch Plains, NJ (US)

Correspondence Address: MERCK AND CO., INC, P O BOX 2000, RAHWAY, NJ, 07065-0907, US

(21) Appl. No.: 10/559,676

(22) PCT Filed: Jun. 16, 2004
(86) PCT No.: PCT/US04/19441

§ 371 Date: Dec. 7, 2005

Related U.S. Application Data
(60) Provisional application No. 60/480,540, filed on Jun. 20, 2003.

(51) Int. Cl. A61K 31/415; A61K 31/365

(52) U.S. Cl. 514/406; 514/471

(57) ABSTRACT

The instant invention is directed to the use of cyclooxygenase-2 selective inhibitors to treat or prevent endometriosis; the use of cyclooxygenase-2 selective inhibitors to prevent, retard and/or reverse the development of endometriotic lesions in patients at risk for the development of such lesions; the use of cyclooxygenase-2 selective inhibitors to reduce the number and/or severity of endometriotoc lesions in patients at risk for the development of such lesion; and the treatment of pain in such patients. Combination therapies are also encompassed.

### BACKGROUND OF THE INVENTION

Many women, approximately 5-10 percent of those in their reproductive years, are afflicted with endometriosis and suffer progressive, disabling dysmenorrhea and pelvic pain around the time of their menses (Brosens, Endometriosis-A Disease Because it is Characterized by Bleeding, Am. J. Obstet. Gynecol. 176:263-7 (1997)). In addition, pelvic pain unassociated with menses may restrict afflicted women to measured participation in athletic and other physical activities, such as dancing and hiking. Through dyspareunia, they suffer not only the pain and often-missed orgasmic fulfillment, but also the doubts of sincerity and the cautious love of their sexual partners, perhaps even marital discord, separation, or infertility. Through relative infertility, they suffer further reductions in self-esteem from the pangs of guilt and failure engendered by struggles to conceive, suffering that adds personal, physical, and economic cost. Often, coital events or pelvic exams produce pelvic aching for hours or even days thereafter.

Selective inhibitors of cyclooxygenase-2 are a sub-class of the class of drugs known as non-steroidal antiinflammatory drugs (NSAIDs). The NSAIDs are active in reducing the prostaglandin-induced pain and swelling associated with the inflammation process but are also active in affecting other prostaglandin-regulated processes not associated with the inflammation process. Thus, use of high doses of most common NSAIDs can produce severe side effects, including life threatening ulcers, that limit their therapeutic potential. An alternative to NSAIDs is the use of corticosteroids, which have even more drastic side effects, especially when long term therapy is involved.

Previous NSAIDs have been found to prevent the production of prostaglandin by inhibiting enzymes in the human arachidonic acid/prostaglandin pathway including the enzyme cyclooxygenase (COX). The recent discovery that there are two isoforms of the COX enzyme, the first, COX-1, being involved with physiological functions and the second, COX-2, being induced in inflamed tissue, has given rise to a new approach. While conventional NSAIDs block both forms of the enzyme, the identification of the inducible COX-2 enzyme associated with inflammation has provided a viable target of inhibition which more effectively reduces inflammation and produces fewer and less drastic side effects. Many compounds which have activity as COX-2 inhibitors have been identified, including rofecoxib (VIOXX®), etoricoxib (ARCOXIA™), celecoxib (CELEBREX®) and valdecoxib (BEXTRA™), and much research continues in this area.

The current invention is directed to the use of cyclooxygenase-2 selective inhibitors for the treatment and prevention of endometriosis. The invention offers advantages over the several lines of therapy that are considered standard treatment for endometriosis, which include nonselective, nonsteroidal anti-inflammatory drugs (NSAIDs), oral contraceptives, gonadotropin releasing hormone (GnRH) agonists/antagonists, and surgical removal of endometriotic implants.

Nonselective NSAIDs are used in the symptomatic treatment of pain associated with endometriosis. The current invention offers several advantages over nonselective NSAIDs. First, the current invention will treat the underlying disease and not just the symptoms through inhibition of aromatase, an enzyme that converts androgens to estrogen and is expressed at abnormally high levels in the endometrium of women with endometriosis. Prostaglandin E2 (PGE2) is the most potent known activator of aromatase (gene and protein activation) and the current invention will serve to inhibit the local concentrations of aromatase in the endometrium through the inhibition of COX-2 and the subsequent inhibition of PGE2. Unlike nonselective NSAIDs, the current invention is able to be used in the perioperative setting and therefore patients undergoing surgical removal of endometriotic lesions can be treated before, during, and immediately after surgery to inhibit the formation of new lesions and to regress lesions not able to be resected during surgery. The current invention has proven superior gastrointestinal safety advantage compared with nonselective NSAIDs.

Progesterone based oral contraceptives are used in the treatment of endometriosis to inhibit pituitary hormones (e.g., GnRH) and ovulation. As monotherapy, this invention offers the advantage that women will not be required to stop treatment for an extended period of time prior to attempting to conceive.

GnRH agonists/antagonists function to diminish the production of two pituitary gonadotropins, leuteinizing hormone (LH) and follicle stimulating hormone (FSH). The resulting hypoestrogenic state leads to endometrial atrophy and amenorrhea. The current invention offers the advantage of inhibiting the local concentration of estrogen through the inhibition of endometrial aromatase as described above rather than inhibiting the systemic expression of estrogen that leads to the common side effects of the GnRH compounds (e.g., bone density loss, hot flashes, weight gain). Estrogen that is produced via aromatase has a positive feedback effect on COX-2, essentially producing a positive feedback loop for local estrogen production in the endometrial tissue with an elevation of COX-2 and PGE-2. The current invention will also serve to directly inhibit COX-2 therefore inhibiting the positive feedback effect.

The current invention offers the advantage over surgical intervention as a noninvasive treatment and as combination or follow-up therapy to surgical intervention.

### SUMMARY OF THE INVENTION

The instant invention is directed to the use of cyclooxygenase-2 selective inhibitors to treat or prevent endometriosis; the use of cyclooxygenase-2 selective inhibitors to prevent, retard and/or reverse the development of endometriotic lesions in patients at risk for the development of such lesions; the use of cyclooxygenase-2 selective inhibitors to reduce the number and/or severity of endometriotoc lesions in patients at risk for the development of such lesion; and the treatment of pain in such patients. Combination therapies are also encompassed.

### BRIEF DESCRIPTION OF THE DRAWINGS

FIG. 1—Mean implant Volume Comparison

FIG. 1 compares the mean volume of rat uterine horn implants in the peritoneal cavity of rats before and after 14 days of administration of a cyclooxygenase-2-selective inhibitor.

FIG. 2—Mean implant Weight Comparison

FIG. 2 compares the in mean weight of rat uterine horn implants in the peritoneal cavity of rats before and after 14 days of administration of a cyclooxygenase-2-selective inhibitor.

### DETAILED DESCRIPTION OF THE INVENTION

In one aspect, the invention is directed to a method for treating or preventing endometriosis in a patient in need of such treatment or prevention, comprising the administration of an effective amount of a cyclooxygenase-2 selective inhibitor to said patient.

In a second aspect, the invention is directed to a method for retarding the development of endometriotic lesions in a patient at risk of the development of said lesions, comprising the administration of an effective amount of cyclooxygenase-2 selective inhibitor to said patient.

In a third aspect, the invention is directed to a method for preventing the development of endometriotic lesions in a patient at risk of the development of said lesions, comprising the administration of an effective amount of a cyclooxygenase-2 selective inhibitor to said patient.

In a fourth aspect, the invention is directed to a method for reversing the development of endometriotic lesions in a patient at risk of the development of said lesions, comprising the administration of an effective amount of cyclooxygenase-2 selective inhibitor to said patient.

In a fifth aspect, the invention is directed to a method of treating pain in patients with endometriosis of a type amenable to hormone therapy, comprising the administration of an effective amount of a cyclooxygenase-2 selective inhibitor to said patient.

In a sixth aspect, the invention is directed to a method of reducing pain in patients with endometriotic lesions, comprising the administration of an effective amount of a cyclooxygenase-2 selective inhibitor to said patient.

In a seventh aspect the invention is directed to a method for reducing number and or severity of endometriotic lesions in a patient having said lesions, comprising the administration of an effective amount of a cyclooxygenase-2 selective inhibitor to said patient.

In an eighth aspect, the invention is directed to a method for retarding the development of endometriotic lesions in a patient with endometriosis of a type amenable to hormonal therapy, comprising the administration of an effective amount of a cyclooxygenase-2 selective inhibitor to said patient.

In a ninth aspect, the invention is directed to a method for preventing the development of endometriotic lesions in a patient with endometriosis of a type amenable to hormonal therapy, comprising the administration of an effective amount of a cyclooxygenase-2 selective inhibitor to said patient.

In a tenth aspect, the invention is directed to a method for reversing the development of endometriotic lesions in a patient with endometriosis of a type amenable to hormonal therapy, comprising the administration of an effective amount of a cyclooxygenase-2 selective inhibitor to said patient.

In an eleventh aspect, the invention is directed to a method of reducing elevated levels of aromatase in women with post-menopausal endometriosis, comprising the administration of an effective amount of a cyclooxygenase-2 selective inhibitor to said patient.

The invention encompasses any of the above methods wherein the cyclooxygenase-2 selective inhibitor is selected from the group consisting of: rofecoxib, etoricoxib, celecoxib, valdecoxib, lumiracoxib, BMS347070, tiracoxib, ABT963, CS502 and GW406381.

The invention also encompasses any of the above methods wherein the cyclooxygenase-2 selective inhibitor is rofecoxib. Within this embodiment rofecoxib is administered at a dose of about 12.5 mg or about 25 mg. Also within this embodiment rofecoxib is orally administered on a once daily basis.

The invention also encompasses any of the above methods wherein the cyclooxygenase-2 selective inhibitor is etoricoxib. Within this embodiment etoricoxib is administered at a dose of about 60 mg, about 90 mg or about 120 mg.

The invention also encompasses any of the above methods wherein the cyclooxygenase-2 selective inhibitor is celecoxib. Within this embodiment celecoxib is administered at a dose of about 100 mg or about 200 mg or about 400 mg.

The invention also encompasses any of the above methods cyclooxygenase-2 selective inhibitor is valdecoxib. Within this embodiment valdecoxib is administered at a dose of about 10 mg or about 20 mg.

The invention also encompasses any of the above methods wherein the cyclooxygenase-2 selective inhibitor is concomitantly or sequentially co-administered with an oral contraceptive. Within this embodiment, the oral contraceptive is selected from the group consisting of: norethindrone, norethindrone acetate, chlormadione acetate, norethynodrel, norgestrel, medroxyprogesterone acetate, megestrol acetate, lynestrenol, quingestrone, ethynodiol acetate, and dimethisterone.

The invention also encompasses any of the above methods wherein the cyclooxygenase-2 selective inhibitor is administered perioperatively or as follow-up therapy to surgical removal of endometriotic implants.

The invention also encompasses any of the above methods wherein the cyclooxygenase-2 selective inhibitor is concomitantly or sequentially co-administered with a GnRH agonist. Within this embodiment the GnRH-agonist is selected from the group consisting of nafarelin acetate, leuprolide acetate, goserelin acetate, and buserelin acetate.

The terms “inhibitor of cyclooxygenase-2”, “cyclooxygenase-2 selective inhibitor” and “COX-2 inhibitor” as used herein embrace compounds which selectively inhibit cyclooxygenase-2 over cyclooxygenase-1, including pharmaceutically acceptable, salts thereof. Employing the human whole blood COX-1 assay and the human whole blood COX-2 assay described in C. Brideau et al, Inflamm. Res. 45: 68-74 (1996), herein incorporated by reference, preferably, the compounds have a cyclooxygenase-2 IC₅₀ of less than about 2 TM in the human whole blood COX-2 assay, yet have a cyclooxygenase-1 IC₅₀ of greater than about 5 TM in the human whole blood COX-1 assay. Also preferably, the compounds have a selectivity ratio of cyclooxygenase-2 inhibition over cyclooxygenase-1 inhibition of at least 10, and more preferably of at least 40. The resulting selectivity may indicate an ability to reduce the incidence of common NSAID-induced side effects, especially erosions and ulceration of the upper gastrointestinal mucosa.

Many cyclooxygenase-2 selective inhibitors are known in the art. Examples of cyclooxygenase-2 selective inhibitors include rofecoxib (VIOXX®, see U.S. Pat. No. 5,474,995, hereby incorporated by reference in its entirety), etoricoxib (ARCOXIA™ see U.S. Pat. No. 5,861,419, hereby incorporated by reference in its entirety), celecoxib (CELEBREX®, see U.S. Pat. No. 5,466,823, hereby incorporated by reference in its entirety), valdecoxib (see U.S. Pat. No. 6,633,272, hereby incorporated by reference in its entirety), parecoxib (see U.S. Pat. No. 5,932,598, hereby incorporated by reference in its entirety), lumiracoxib (PREXIGE®, Novartis), BMS347070 (Bristol Myers Squibb), tiracoxib or JTE522 (Japan Tobacco), ABT963 (Abbott), CS502 (Sankyo) and GW406381 (GlaxoSmithKline).

The compounds of use in this invention may have one or more chiral centers and the present compounds may occur as racemates, racemic mixtures and as individual diasteriomers or enantiomers with all such isomeric forms and mixtures thereof being included within the scope of this invention. Furthermore, some of the crystalline forms for compounds of the present invention may exist as polymorphs and as such are intended to be included in the present invention. In addition, some of the compounds of the instant invention may form solvates with water or common organic solvents. Such solvates and hydrates, as well as anhydrous compositions, are encompassed within the scope of this invention. Some of the compounds described herein may contain olefinic double bonds, and unless specified otherwise, are meant to include both E and Z geometric isomers.

The cyclooxygenase-2 selective inhibitors that may be used with this invention encompass all pharmaceutically acceptable salt forms of the compounds. Examples of such salt forms of the cyclooxygenase-2 selective inhibitors include but are not limited to salts derived from inorganic bases including aluminum, ammonium, calcium, copper, ferric, ferrous, lithium, magnesium, manganic salts, manganous, potassium, sodium, zinc, and the like. Particularly preferred are the ammonium, calcium, magnesium, potassium, and sodium salts. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like.

The term “concomitantly administering” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

The term “sequentially administering” means administering the agents at separately staggered times. Thus, agents can be sequentially administered such that the beneficial pharmaceutical effect are realized by the patient at substantially the same time. Thus, for example, if a cyclooxygenase-2 selective inhibitors and other agent are both administered on a once a day basis, the interval of separation between sequential administration of the two agents can be up to twelve hours apart.

The term “therapeutically effective amount” is intended to mean that amount of a drug or pharmaceutical agent that will elicit the biological or medical response of a tissue, a system, animal or human that is being sought by a researcher, veterinarian, medical doctor or other clinician.

The term “patient” includes mammals, especially humans, who take a selective COX-2 inhibitor for any of the uses described herein. Administering of the drug to the patient includes both self-administration and administration to the patient by another person.

Conventional doses of cyclooxygenase-2 selective inhibitors may be used with the present invention. Such amounts are well known in the art and described, for example, in the PDR. Typically, suitable levels will be about 5 to 500 mg per day, preferably 10 to 200 mg per day, and especially 10 to 100 mg/kg per day. The compound may be administered on a regimen of up to 6 times per day, preferably 1 to 4 times per day, and especially once per day.

Additional active agents may be used in combination with the COX-2 inhibitor in a single dosage formulation, or may be administered to the patient in a separate dosage formulation, which allows for concurrent or sequential administration. One or more additional active agents may be administered with the COX-2 inhibitor. The additional active agent or agents include oral contraceptives and GnRH agonists as listed above.

The additional active agents described above which may be employed along with the COX-2 inhibitor, for example, in amounts as indicated in the PDR or in amounts as indicated in the reference disclosures, as appropriate.

The active agents employed in the instant combination therapy can be administered in such oral forms as tablets, capsules (each of which includes sustained release or timed release formulations), pills, powders, granules, elixirs, tinctures, suspensions, syrups, and emulsions. The instant invention includes the use of both oral rapid-release and time-controlled release pharmaceutical formulations. A particular example of an oral time-controlled release pharmaceutical formulation is described in U.S. Pat. No. 5,366,738. Oral formulations are preferred. Such pharmaceutical compositions are known to those of ordinary skill in the pharmaceutical arts; for example, see Remington's Pharmaceutical Sciences, Mack Publishing Co., Easton, Pa.

In the methods of the present invention, the active agents are typically administered in admixture with suitable pharmaceutical diluents, excipients or carriers (collectively referred to herein as “carrier” materials) suitably selected with respect to the intended form of administration, that is, oral tablets, capsules, elixirs, syrups and the like, and consistent with conventional pharmaceutical practices.

For instance, for oral administration in the form of a tablet or capsule, the active drug component can be combined with a non-toxic, pharmaceutically acceptable, inert carrier such as lactose, starch, sucrose, glucose, modified sugars, modified starches, methyl cellulose and its derivatives, dicalcium phosphate, calcium sulfate, mannitol, sorbitol and other reducing and non-reducing sugars, magnesium stearate, steric acid, sodium stearyl fumarate, glyceryl behenate, calcium stearate and the like. For oral administration in liquid form, the drug components can be combined with non-toxic, pharmaceutically acceptable inert carrier such as ethanol, glycerol, water and the like. Moreover, when desired or necessary, suitable binders, lubricants, disintegrating agents and coloring and flavoring agents can also be incorporated into the mixture. Stabilizing agents such as antioxidants (BHA, BHT, propyl gallate, sodium ascorbate, citric acid) can also be added to stabilize the dosage forms. Other suitable components include gelatin, sweeteners, natural and synthetic gums such as acacia, tragacanth or alginates, carboxymethylcellulose, polyethylene glycol, waxes and the like.

The active drugs can also be administered in the form of liposome delivery systems, such as small unilamellar vesicles, large unilamellar vesicles and multilamellar vesicles. Liposomes can be formed from a variety of phospholipids, such as cholesterol, stearylamine or phosphatidylcholines.

Active drug may also be delivered by the use of monoclonal antibodies as individual carriers to which the compound molecules are coupled. Active drug may also be coupled with soluble polymers as targetable drug carriers. Such polymers can include polyvinyl-pyrrolidone, pyran copolymer, polyhydroxy-propyl-methacrylamide-phenol, polyhydroxy-ethyl-aspartamide-phenol, or polyethyleneoxide-polylysine substituted with palmitoyl residues. Furthermore, active drug may be coupled to a class of biodegradable polymers useful in achieving controlled release of a drug, for example, polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels.

Although the active agents of the present method may be administered in divided doses, for example two or three times daily, a single daily dose of the selective COX-2 inhibitor is preferred, where approved.

The instant invention also encompasses a process for preparing a pharmaceutical composition comprising combining the cyclooxygenase-2 selective inhibitor with a pharmaceutically acceptable carrier, as well as the pharmaceutical composition which is made by combining the cyclooxygenase-2 selective inhibitor with a pharmaceutically acceptable carrier.

The medicament or pharmaceutical combination comprised of the COX-2 inhibitor may also be prepared with one or more additional active agents, such as those described supra.

The Utility of selective COX-2 inhibitors in the management of endometriosis is illustrated by the following assay:

Rat Model for Endometriosis:

In this model 5×5 mm sections of rat uterine hom are implanted in the peritoneal cavity (extra uterine endometrial wall) of subject rats. Four weeks after transplantation of the endometrial tissues, rats were anesthetized with Telazol (20 mg/kg, IP, Fort Dodge Animal Health, Fort Dodge, Iowa), Oxymorphone (0.2 mg/kg SC, Henry Schein, Melville, N.Y.) and immobilized on a standard rat operating board. The surgical site was shaved and cleaned using three cycles of Betadine/alcohol or DuraPrep® (3M, St. Paul, Minn.). Using aseptic technique, an incision was made through the skin, SC and muscle layers. The growth of the explant was evaluated for size and acceptance/rejection. On gross examination, the endometrial implants appeared as uni-loculated (occasional as multilobulated) cystic masses filled with clear/light yellowish or dark brown serous fluid. In some instances, at the time of 2nd laparotomy, partial or complete adhesions from connective tissue, mesentry, and bowel to the implants were noted. In those instances, a careful separation was executed, avoiding the rupture the implants. While having a clear vision of the implants, the length, width and height of the implant was measured using a sterile Jameson Caliper (BRI, Rockville, Md.), and documented. The abdominal incisions were closed by using 4-0 chromic gut (Ethicon). The rats were observed until fully ambulatory.

More animals than required for the study were used for surgery. After the 2^{nd} laparotomy, animals were assigned to control or treated groups in an attempt to equalize the endometrium implant size between groups. The rats bearing implant with clear/light yellowish fluid were used in study group selection. The rats bearing implant with dark brown fluid considered as hemorrhagic solid mass were excluded from the study.

Treatment with selected drug therapy started after 1 day of post-surgical recovery. Rats were given the test compound, the cyclooxygenase-2 selective inhibitor Compound A, or vehicle alone or Raloxifene (10 mpk) or Lupron Depot (1 mpk) for a period of 14 days. At the end of the 14-day treatment period, the animals were euthanized at pro-estrus cycle which was confirmed by vaginal smear. Necropsy involved opening the abdomen and evaluating the implant size. The implant and right uterine horn were excised for wet weighing.

Optionally the test rat implants may be evaluated for estrogen (e.g. by radio immune assay); for COX-2 (e.g. by western blot); Prostaglandin E2 (e.g. by enzyme immunoassay [EIA]).

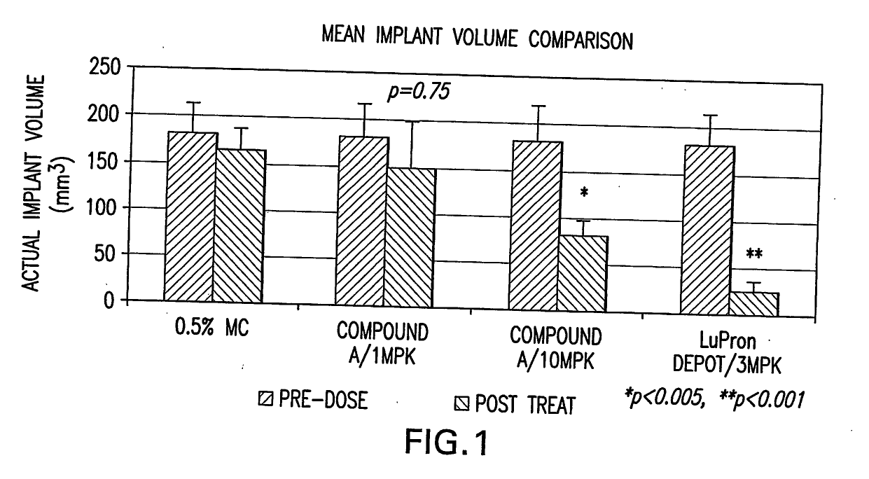

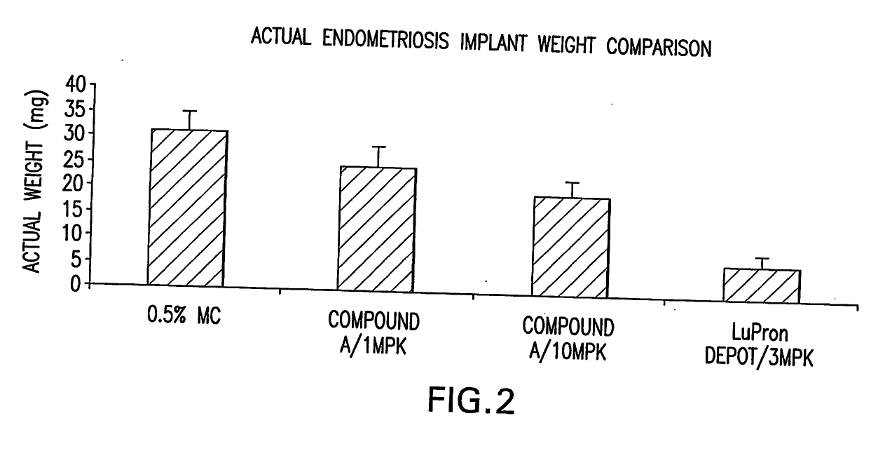

As illustrated in FIGS. 1 and 2, administration of 10 mpk (BD by oral gavage) of the cyclooxygenase-2 selective inhibitor Compound A has been shown to reduce implant volume and weight to a significant degree at the 95 percent confidence level. In this comparison 1 mpk mpk (BID by oral gavage) was not significant at the 95 percent confidence level. Compound A has the following structure: 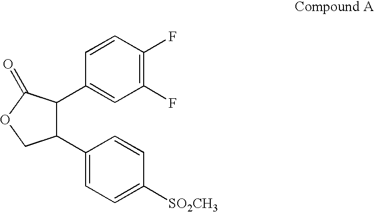
Compound A and methods of synthesis are disclosed in U.S. Pat. No. 5,474,995, granted Dec. 12, 1995.
Examples of dosage formulations suitable for use in practicing the instant invention follow. Additionally, cyclooxygenase-2 selective inhibitors are commercially available, e.g., rofecoxib (VIOXX®), etoricoxib (ARCOXIA™), celecoxib (CELEBREX®) and valdecoxib (BEXTRA™).

### EXAMPLE 1

Wet granulated tablet composition

| Amount per tablet | | Ingredient |
|---|---|---|
| 25 | mg | COX-2 Inhibitor |
| 79.7 | mg | Microcrystalline cellulose |
| 79.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### EXAMPLE 1A

Wet granulated tablet composition

| Amount per tablet | | Ingredient |
|---|---|---|
| 12.5 | mg | COX-2 Inhibitor |
| 86 | mg | Microcrystalline cellulose |
| 86 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 1B

Wet granulated tablet composition

| Amount per tablet | | Ingredient |
|---|---|---|
| 10 | mg | COX-2 Inhibitor |
| 87.2 | mg | Microcrystalline cellulose |
| 87.2 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 1C

Wet granulated tablet composition

| Amount per tablet | | Ingredient |
|---|---|---|
| 5 | mg | COX-2 Inhibitor |
| 89.7 | mg | Microcrystalline cellulose |
| 89.7 | mg | Lactose monohydrate |
| 6 | mg | Hydroxypropyl cellulose |
| 8 | mg | Croscarmellose sodium |
| 0.6 | mg | Iron oxide |
| 1 | mg | Magnesium stearate |

### EXAMPLE 2

Directly compressed tablet composition

| Amount per tablet | | Ingredient |
|---|---|---|
| 25 | mg | COX-2 Inhibitor |
| 106.9 | mg | Microcrystalline cellulose |
| 106.9 | mg | Lactose anhydrate |
| 7.5 | mg | Crosmellose sodium |
| 3.7 | mg | Magnesium stearate |

Tablet dose strengths of between 5 and 125 mg can be accomodated by varying total tablet weight, and the ratio of the first three ingredients. Generally it is preferable to maintain a 1:1 ratio for microcrystalline cellulose:lactose monohydrate.

### EXAMPLE 2A

Directly compressed tablet composition

| Amount per tablet | | Ingredient |
|---|---|---|
| 12.5 | mg | COX-2 Inhibitor |
| 113.2 | mg | Microcrystalline cellulose |
| 113.2 | mg | Lactose anhydrate |
| 7.5 | mg | Croscarmellose sodium |
| 3.7 | mg | Magnesium stearate |

### EXAMPLE 2B

Directly compressed tablet composition

| Amount per tablet | | Ingredient |
|---|---|---|
| 10 | mg | COX-2 Inhibitor |
| 42.5 | mg | Microcrystalline cellulose |
| 42.5 | mg | Lactose anhydrate |
| 4 | mg | Croscarmellose sodium |
| 1 | mg | Magnesium stearate |

### EXAMPLE 2C

Directly compressed tablet composition

| Amount per tablet | | Ingredient |
|---|---|---|
| 5 | mg | COX-2 Inhibitor |
| 45 | mg | Microcrystalline cellulose |
| 45 | mg | Lactose anhydrate |
| 4 | mg | Croscarmellose sodium |
| 1 | mg | Magnesium stearate |

### EXAMPLE 3

Hard gelatin capsule composition

| Amount per capsule | | Ingredient |
|---|---|---|
| 25 | mg | COX-2 Inhibitor |
| 37 | mg | Microcrystalline cellulose |
| 37 | mg | Lactose anhydrate |
| 1 | mg | Magnesium stearate |
| 1 | capsule | Hard gelatin capsule |

Capsule dose strengths of between 1 and 50 mg can be accomodated by varying total fill weight, and the ratio of the first three ingredients. Generally it is preferable to maintain a 1:1 ratio for microcrystalline cellulose:lactose monohydrate.

### EXAMPLE 4

Oral solution

| Amount per 5 mL dose | Ingredient |
|---|---|
| 50 mg to 5 mL with Polyethylene oxide 400 | COX-2 Inhibitor |

Solution dose strengths of between 1 and 50 mg/5 mL can be accomodated by varying the ratio of the two ingredients.

### EXAMPLE 5

Oral suspension

| Amount per 5 mL dose | | Ingredient |
|---|---|---|
| 101 | mg | COX-2 Inhibitor |
| 150 | mg | Polyvinylpyrrolidone |
| 2.5 | mg | Poly oxyethylene |
| | | sorbitan monolaurate |
| 10 | mg | Benzoic acid |
| to 5 | mL | |

with sorbitol solution (70%)

Suspension dose strengths of between 1 and 50 mg/5 ml can be accomodated by varying the ratio of the first two ingredients.

### EXAMPLE 6

Intravenous infusion

| Amount per 200 mL dose | | Ingredient |
|---|---|---|
| 1 | mg | COX-2 inhibitor |
| 0.2 | mg | Polyethylene oxide 400 |
| 1.8 | mg | Sodium chloride |
| to 200 | mL | Purified water |

While the invention has been described and illustrated with reference to certain particular embodiments thereof, those skilled in the art will appreciate that various changes, modifications and substitutions can be made therein without departing from the spirit and scope of the invention. For example, effective dosages other than the particular dosages as set forth herein above may be applicable as a consequence of variations in the responsiveness of the mammal being treated for any of the indications for the active agents used in the instant invention as indicated above. Likewise, the specific pharmacological responses observed may vary according to and depending upon the particular active compound selected or whether there are present pharmaceutical carriers, as well as the type of formulation and mode of administration employed, and such expected variations or differences in the results are contemplated in accordance with the objects and practices of the present invention. It is intended, therefore, that the invention be defined by the scope of the claims which follow and that such claims be interpreted as broadly as is reasonable.

1. A method for treating or preventing endometriosis in a patient in need of such treatment or prevention, comprising the administration of an effective amount of a cyclooxygenase-2 selective inhibitor to said patient.

2. A method for retarding the development of endometriotic lesions in a patient at risk of the developing said lesions, comprising the administration of an effective amount of a cyclooxygenase-2 selective inhibitor to said patient, in accordance with claim 1.

3. A method for preventing the development of endometriotic lesions in a patient at risk of the developing said lesions, comprising the administration of an effective amount of a cyclooxygenase-2 selective inhibitor to said patient, in accordance with claim 1.

4. A method for reversing the development of endometriotic lesions in a patient at risk of the developing said lesions, comprising the administration of an effective amount of a cyclooxygenase-2 selective inhibitor to said patient, in accordance with claim 1.

5. A method for reducing number or severity of endometriotic lesions in a patient having said lesions, comprising the administration of an effective amount of a cyclooxygenase-2 selective inhibitor to said patient, in accordance with claim 1.

6. A method for retarding the development of endometriotic lesions in a patient with endometriosis of a type amenable to hormonal therapy, comprising the administration of an effective amount of a cyclooxygenase-2 selective inhibitor to said patient, in accordance with claim 1.

7. A method for preventing the development of endometriotic lesions in a patient with endometriosis of a type amenable to hormonal therapy, comprising the administration of an effective amount of a cyclooxygenase-2 selective inhibitor to said patient, in accordance with claim 1.

8. A method for reversing the development of endometriotic lesions in a patient with endometriosis of a type amenable to hormonal therapy, comprising the administration of an effective amount of a cyclooxygenase-2 selective inhibitor to said patient, in accordance with claim 1.

9. A method of inhibiting aromatase in a patient at risk of having endometriotic lesions, comprising the administration of an effective amount of a cyclooxygenase-2 selective inhibitor to said patient, in accordance with claim 1.

10. A method of reducing elevated levels of aromatase in a patient at risk of having endometriotic lesions, comprising the administration of an effective amount of a cyclooxygenase-2 selective inhibitor to said patient, in accordance with claim 1.

11. A method of reducing elevated levels of aromatase in a patient with post menapausal endometriosis, comprising the administration of an effective amount of a cyclooxygenase-2 selective inhibitor to said patient, in accordance with claim 1.

12. The method according to claim 1 wherein the cyclooxygenase-2 selective inhibitor is selected from the group consisting of: rofecoxib, etoricoxib, celecoxib, valdecoxib, lumiracoxib, BMS347070, tiracoxib, ABT963, CS502 and GW406381.

13. The method according to claim 1 wherein the cyclooxygenase-2 selective inhibitor is rofecoxib.

14. (canceled)

15. (canceled)

16. The method according to claim 1 wherein the cyclooxygenase-2 selective inhibitor is etoricoxib.

17. (canceled)

18. The method according to claim 1 wherein the cyclooxygenase-2 selective inhibitor is celecoxib.

19. (canceled)

20. The method according to claim 1 wherein the cyclooxygenase-2 selective inhibitor is valdecoxib.

21. (canceled)

22. The method according to claim 1, wherein the cyclooxygenase-2 selective inhibitor is concomitantly or sequentially co-administered with an oral contraceptive.

23. The method according to claim 22 wherein the oral contraceptive is selected from the group consisting of: norethindrone, norethindrone acetate, chlormadione acetate, norethynodrel, norgestrel, medroxyprogesterone acetate, megestrol acetate, lynestrenol, quingestrone, ethynodiol acetate, and dimethisterone.

24. The method according to claim 1 wherein the cyclooxygenase-2 selective inhibitor is administered perioperatively or as follow-up therapy to surgical removal of endometriotic implants.

25. The method according to claim 1, wherein the cyclooxygenase-2 selective inhibitor is concomitantly or sequentially co-administered with a GnRH agonist.

26. The method according to claim 25 wherein the GnRH-agonist is selected from the group consisting of nafarelin acetate, leuprolide acetate, goserelin acetate, and buserelin acetate.
